(19) United States
(12) Patent Application Publication
(10) Pub. No.: US 2025/0261845 A1
(43) Pub. Date: Aug. 21, 2025

(54) UTILIZING FIBER BUNDLES AND HOMOGENIZERS FOR UNIFORM ILLUMINATION OF OPHTHALMOLOGY SURGICAL MICROSCOPES

(71) Applicant: Alcon Inc., Fribourg (CH)

(72) Inventors: Pemba Lama, Orange, CA (US); Ronald T. Smith, Irvine, CA (US); John Park, Irvine, CA (US)

(21) Appl. No.: 19/057,777

(22) Filed: Feb. 19, 2025

Related U.S. Application Data
(60) Provisional application No. 63/555,984, filed on Feb. 21, 2024.

(51) Int. Cl. A61B 3/00; A61B 3/13

(52) U.S. Cl. CPC A61B 3/0008; A61B 3/13

(57) ABSTRACT

Optical systems for forming uniform illumination are disclosed. The optical system includes a light source coupled to a plurality of first light carriers providing a plurality of light intensities and a second light carrier coupled to the plurality of first light carriers. The second light carrier spatially homogenizes the plurality of light intensities to form a homogenized light intensity. The optical system further includes an illumination module coupled to the second light carrier and operable to receive the homogenized light intensity.

### INTRODUCTION

This section provides information to facilitate a better understanding of the various aspects of the disclosure. It should be understood that the statements in this section of this document are to be read in this light, and not as admissions of prior art.

Ophthalmic microscopes are generally designed to provide high contrast and detailed imaging of all regions of an eye. Illumination systems of ophthalmic microscopes provide light to illuminate regions of the eye during an eye procedure at a working area (e.g., the corneal plane). While these illumination systems may provide illumination to the eye, they suffer from having a non-uniform distribution of light and provide poor color mixing at the region being viewed (e.g., the corneal plane). In addition, these systems generally experience bending loss in optical waveguides when the ophthalmic microscope arm and/or optical head are moved.

### SUMMARY

This summary is provided to introduce a selection of concepts that are further described below in the Detailed Description. This summary is not intended to identify key or essential features of the claimed subject matter, nor is it to be used as an aid in limiting the scope of the claimed subject matter.

Certain embodiments herein pertain to an optical system including a light source coupled to a plurality of first light carriers providing a plurality of light intensities and a second light carrier coupled to the plurality of first light carriers. The second light carrier spatially homogenizes the plurality of light intensities to form a homogenized light intensity. The optical system further includes an illumination module coupled to the second light carrier and is operable to receive the homogenized light intensity.

Certain embodiments herein pertain to an optical system including a light source coupled to a light carrier. The light carrier spatially homogenizes a plurality of light intensities to form a homogenized light intensity. The optical system further includes an optical head having an illumination module coupled to the light carrier and operable to receive the homogenized light intensity. The optical head is spaced apart from the light source.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates an example microscope, according to aspects of the disclosure.

FIG. 2 illustrates an example optical system operable to be coupled to, or integrated with, a microscope (e.g., the microscope of FIG. 1), according to aspects of the disclosure.

FIG. 3A illustrates a first end of a randomized fiber optic bundle, according to aspects of the disclosure.

FIG. 3B illustrates a second end of the randomized fiber optic bundle, according to aspects of the disclosure.

FIG. 4A illustrates a fiber optic bundle having a split-rod, according to aspects of the disclosure.

FIG. 4B illustrates a bifurcated fiber optic bundle, according to aspects of the disclosure.

FIG. 4C illustrates a fiber optic bundle having a fiber optic splitter, according to aspects of the disclosure.

FIG. 5 illustrates an example embodiment of a second light carrier, according to aspects of the disclosure.

FIG. 6 illustrates an example embodiments of a second light carrier having a hexagonal shape, according to aspects of the disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Various ophthalmic microscopes may utilize either a single core optical fiber or liquid light guides (LLGs) as a waveguide medium in the microscope. Although the light transmission from LLGs are decent, there are several drawbacks with them. For example, LLGs have poor mechanical reliability, especially at the shipping temperature, which is generally less than −40° C. and sometimes more than 70° C. Additionally, the seals that protect the liquid inside the LLGs tend to break, which can result in the formation of bubbles within the LLGs. Once the seal of an LLG is broken, the damage is irreversible. Furthermore, the lifetime of an LLG is generally only around 2 to 4 years, and therefore, LLGs are not suitable for long-life equipment. Another problem with LLGs is that the allowable bend radius is large, preventing opto-mechanical engineers from designing a flexible cable routing path for ophthalmic microscopes.

In certain ophthalmic microscopes, there may be a fiber optic bundle that propagates light from a remotely positioned source, for example, a xenon source, to its illumination optics. However, these fiber optic bundles are not randomized, and there is no beam homogenizing component, or homogenizer, after the fiber optic bundle. As such, the light intensity is not evenly distributed and “hot spots” of higher light intensity are noticeable in the middle of the projected beam at the corneal plane.

As used herein, the term “randomized”, when referring to fiber optic bundles or other light carriers, refers to the placement of light carriers within a ferrule, sheathing, or other holder/containment device (e.g., purposeful placement of one or more fibers of the fiber optic bundle). For example, with respect to randomized fiber optic bundles, relative end positions of one or more fibers within the randomized fiber optic bundle may change from its input end position to another position at the output end of the fiber optic bundle. As such, light waves may enter at one position of the randomized fiber optic bundle and exit at a different position.

Stated differently, light entering into the input end of the randomized fiber optic bundle (or other light carrier) may or may not have the same corresponding position at the output end of the randomized fiber optic bundle, relative to each fiber optic within the randomized fiber optic bundle. As an example, if light enters into the randomized fiber optic bundle at a top portion of the input end, the light will propagate through a fiber optic of the randomized fiber optic bundle and may exit at a bottom portion of the output end of the randomized fiber optic bundle. In this example, the fiber optic had a top position in the randomized fiber optic bundle at the input end and a bottom position in the randomized fiber optic bundle at the output end. It should be noted that, as used herein, randomized fiber optic bundles and/or light carriers may or may not have a one-to-one mapping from an input end to an output end. However, in certain embodiments, a randomized fiber optic bundle and/or light carrier may have substantially no one-to-one mappings.

Accordingly, certain embodiments herein pertain to utilizing a light carrier that may include, for example, a fiber optic bundle in conjunction with a light beam homogenizer to produce a spatially homogenized beam for ophthalmic illumination applications. In various embodiments disclosed herein, various light-emitting diodes (LEDs), for example red, green, and blue LEDs, are mixed to obtain a white light, and may be remotely located in the microscope system. In certain embodiments, a module that contains a randomized fiber optic bundle, in combination with a clad-rod homogenizer, may be used to route the light beam to the corneal plane. The clad-rod homogenizer may spatially homogenize the light intensity and mix the color of the LEDs.

In certain embodiments, the systems described herein may have various advantages over optical systems currently available. For example, one advantage of using a randomized fiber optic bundle as a light carrier is that the routing may be more flexible when compared to a single core optical fiber. The remote light source (e.g., red, blue, and green LEDs), the randomized fiber optic bundle, and the clad-rod homogenizer may be integrated via simple, and standard, SubMiniature version A (SMA) connectors.

In another example, randomized fiber optic bundles discussed herein may contain tiny borosilicate fibers with a core diameter in a range of ˜15 to 100 μm, ˜20 to 70 μm, and/or ˜30 to 70 μm. In certain embodiments, the core diameter may be ˜50 μm. Approximately 1,000 to 2,000 of these fibers may be packed to form an active area in a range of ˜0.5 to 4 mm (millimeters). In certain embodiments, 1,400 of these fibers are packed to form an active area of ˜2 mm and are randomly arranged. The randomization of the individual fibers can help in spatial intensity homogenization, especially when an input light is spatially quasi-Gaussian (e.g., LED sources are spatially quasi-Gaussian in nature, with a central hot spot that monotonically decreases in a radial fashion towards the edge of the beam). Additionally, fiber bending macro-level loss is directly proportional to the fiber core diameter, and for a given bend radius, larger core diameter fibers have higher bending loss than the smaller core diameter fibers. Therefore, in certain embodiments, the use of micron-sized fibers (e.g., ˜15 to 100 μm core diameters) allows the fiber optic bundle to be bent as small as ˜2.5 to 10 mm in short term and ˜15 to 35 mm in long term bend radii, thereby providing flexibility in routing the fiber optic bundle.

In various embodiments, as further advantages, various optical systems described herein enable light sources, light engines, and/or illumination sources to be mounted anywhere in the optical system, as the light can be propagated through the flexible optical fibers. The fibers may be fused at the input end to achieve a higher packing fraction and epoxied at the output end. A higher packing fraction can provide higher transmittance. In certain applications, fusing only the input end may be necessary. However, both ends may be fused or epoxied in some embodiments. In certain embodiments, fused fiber optic bundles may provide more than ˜20% higher transmission compared to unfused, or epoxied fiber optic bundles. Moreover, in various embodiments, superior spatial homogenization and color mixing can be achieved by adding a homogenizer at the output end.

In certain embodiments, a second light channel (e.g., a homogenizer) may contain a thin cladding layer which protects the core material from touching other material. Taking advantage of the cladding layer, the homogenizer can be assembled in a unique male-female SMA adapter, making it much easier to couple with the fiber optic bundle and integrate with the light source, that is remotely placed, and into the existing illumination system.

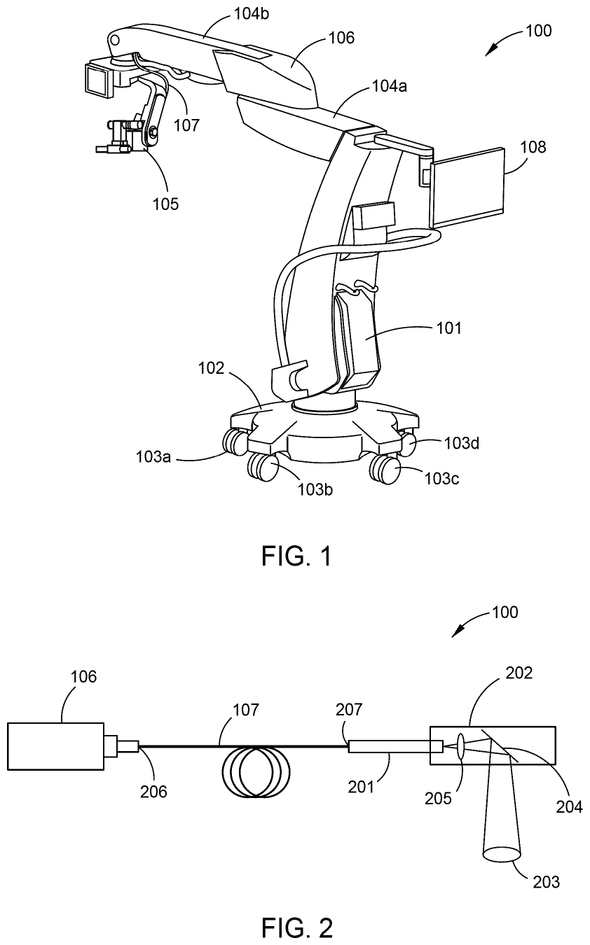

FIGS. 1-2 illustrates an example microscope system 100 according to aspects of the disclosure. FIG. 1 is a perspective view of the microscope system 100, while FIG. 2 illustrates certain operational aspects of the microscope system 100. In certain embodiments, microscope system 100 may be an ophthalmic microscope system. For clarity, FIGS. 1-2 will be described collectively.

As shown in FIG. 1, microscope system 100 can include a base 101 coupled to a floor stand 102 having wheels 103a-d (collectively referred to as wheels 103). While FIG. 1 illustrates microscope system 100 having a floor stand 102, in certain embodiments, the microscope system 100 may be mounted on a table, cabinet, wall, or the floor. In some embodiments, microscope system 100 may be a tabletop microscope system. Base 101 is movably coupled to a first arm 104a that is coupled to a joint assembly 110. Joint assembly 110 is coupled to a second arm 104b operable to movably couple to an optical head 105 (e.g., a microscope). In some embodiments, the first arm 104a and the second arm 104b may include a plurality of arms connected together via arm joints such that the optical head 105 may be moved to various locations around the base 101 (e.g., positioned over an eye to view a corneal plane).

Microscope system 100 includes a light source 106 (shown in FIG. 2) connected to an illumination module 202 (shown in FIG. 2) via a plurality of first light carriers 107 (e.g., through second arm 104b), as will be described in further detail below. In some embodiments, the light source 106 is spaced apart from the optical head 105. In some embodiments, the light source 106 may be near and/or connected to the first arm 104a and/or the second arm 104b. For example, in some embodiments, the light source 106 is housed within the joint assembly 110. In various embodiments, the light source is coupled to the base 101. Microscope system 100 includes a display screen 108 operable to output images of a viewing area of the microscope system 100. Display screen 108 may include, for example, a liquid crystal display (LCD), a plasma display, an LED display, organic LED displays, and the like. While FIG. 1 illustrates the microscope system 100 having one display screen 108, in certain embodiments, the display screen 108 may include a plurality of display screens 108.

With particular reference to FIG. 2, light source 106 is coupled to the plurality of first light carriers 107 via, for example, a SMA connector. In some embodiments, the light source 106 may include, for example, an ultraviolet light source, a laser, an infrared (IR) light source, a near-IR light source, or one or more LEDs. In certain embodiments, the one or more LEDs may be a red LED, a green LED, a blue LED, and combinations of the same and like.

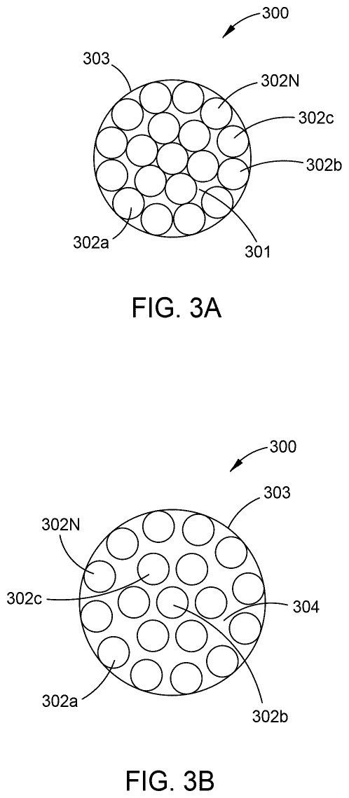

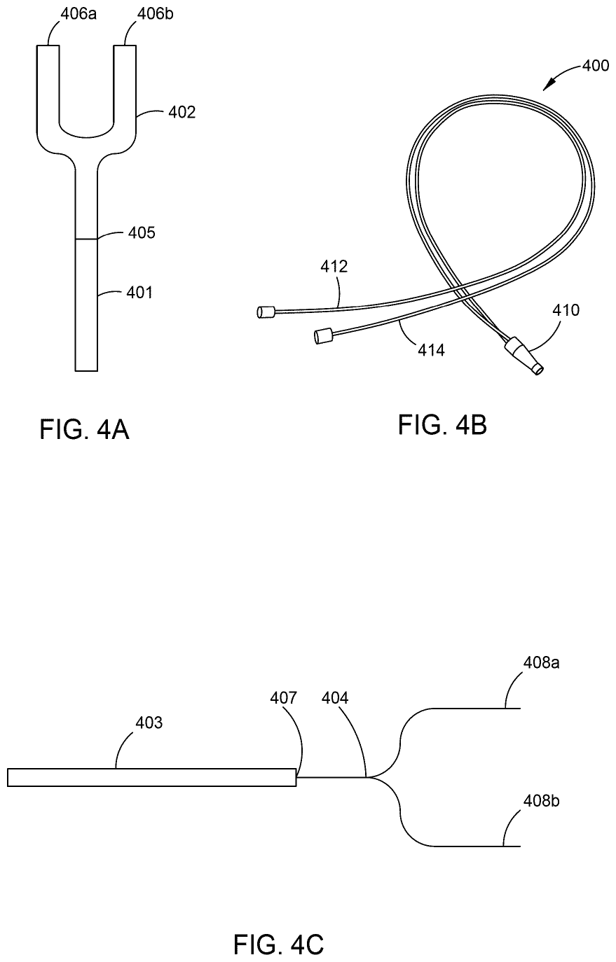

In certain embodiments, the plurality of first light carriers 107 includes a randomized fiber optic bundle. In some embodiments, the plurality of first light carriers 107 may be any light carrier operable to propagate light. In some embodiments, the plurality of first light carriers 107 includes a first end 206 and a second end 207. In some embodiments, the first end 206 may include fused optical fibers (e.g., of a randomized fiber optic bundle). In some embodiments, the second end 207 may include epoxied optical fibers (e.g., of a randomized fiber optic bundle). In some embodiments, the plurality of first light carriers may include, for example, a fiber optic bundle having a split rod, a bifurcated fiber optic bundle, or a fiber optic bundle having one or more fiber splitters. In the illustrated embodiment, the first end 206 is a light input end and the second end 207 is a light output end. Examples of the plurality of first light carriers 107 will be described in greater detail relative to FIGS. 3A-3B and 4A-4C.

In the embodiment shown in FIGS. 1-2, a second light carrier 201 (e.g., a homogenizer) is coupled to the plurality of first light carriers 107 such that the plurality of first light carriers 107 provide a plurality of light intensities to the second light carrier 201. In some embodiments, the plurality of first light carriers 107 is coupled to the second light carrier 201 via a SMA connector. In general, the second light carrier 201 spatially homogenizes the plurality of light intensities from the plurality of first light carriers 107 to form a homogenized light intensity. In certain embodiments, second light carrier 201 mixes light color from the plurality of light intensities. In certain embodiments, the plurality of first light carriers 107 integrates with the second light carrier 201.

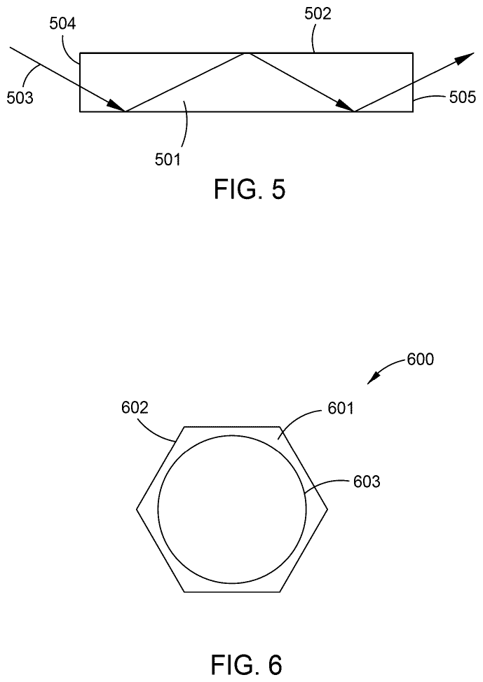

In certain embodiments, the second light carrier 201 may include a cylindrical clad-rod borosilicate. In some embodiments, the second light carrier 201 includes a medium therein, such as, for example, air. In various embodiments, the second light carrier 201 includes a hexagonal rod having a round aperture at the distal end. In some embodiments, the second light carrier 201 may have, for example, a cylindrical shape, a hexagonal shape, a rectangular shape, or a square shape. In certain embodiments, the second light carrier 201 has a cladding surrounding the core of second light carrier 201. In some embodiments, the second light carrier 201 has a cladding having a mirrored surface surrounding the second light carrier 201. Examples of the second light carrier 201 will be described in greater detail relative to FIGS. 5 and 6.

The illumination module 202 is coupled to the second light carrier 201 and is operable to receive the homogenized light intensity and may project the homogenized light intensity to a viewing area 203. In some embodiments, the illumination module 202 is coupled to the second light carrier 201 via a SMA connector. In certain embodiments, the viewing area 203 may include, without limitation, a portion of an eye, such as the cornea, the iris, the pupil, the lens, the ciliary muscle, or other regions of the eye. In certain embodiments, the viewing area is the corneal plane of an eye.

As illustrated in FIG. 2, the illumination module 202 may also include a beam splitter 204 and/or a lens 205. In certain embodiments, beam splitter 204 may be integrated into the illumination module 202 to split the homogeneous light intensity (e.g., in the form of light waves) into multiple paths. In such embodiments, the beam splitter 204 may be utilized to coaxially illuminate the viewing area 203 (e.g., a corneal plane).

In certain embodiments, the beam splitter 204 may be a fold mirror. In such embodiments, the fold mirror may be closer to the second light carrier 201, outside of the microscope's viewing path relative to beam splitter 204 of FIG. 2. In certain embodiments, the fold mirror may be angled such that the beam is projected onto the corneal plane at an oblique angle relative to vertical (e.g.,) 6-12°. This may, in some embodiments, provide general illumination of an eye region where strong red reflex is unwanted.

In certain embodiments, the illumination module 202 may be distinct from other components of the microscope system 100. In such embodiments, an objective lens of the optical head 105 may be located above the illumination module 202 (such that the illumination module 202 is arranged downstream, or below, the objective lens) to image the corneal plane into a camera or to eyes through a binocular. Alternatively, in certain embodiments, the illumination module may be arranged within, and/or integrated with, microscope system 100, for example, above the objective lens of the microscope system 100.

FIGS. 3A and 3B illustrate an example of a randomized fiber optic bundle 300. In certain embodiments, the randomized fiber optic bundle 300 may serve, for example, as the plurality of first light carriers 107 of FIGS. 1-2. FIG. 3A shows a first end 301 of the randomized fiber optic bundle 300. FIG. 3B shows a second end 304 of the randomized fiber optic bundle 300. FIGS. 3A and 3B are described together for clarity.

In certain embodiments, the first end 301 and the second end 304 correspond to the first end 206 and the second end 207, respectively, of FIG. 2. In some embodiments, the first end 301 is a light input end and the second end 304 is a light output end. The randomized fiber optic bundle 300 includes optical fibers 302a, 302b, 302c . . . 302N (collectively referred to as optical fibers 302) enclosed within sheathing 303. FIGS. 3A and 3B show a randomization of optical fibers 302. In certain embodiments, one or more fibers of the optical fibers 302 may have a substantially different placement within the sheathing 303 at the first end 301 relative to the second end 304 (e.g., optical fibers 302b and 302c). In some embodiments, one or more fibers of the optical fibers 302 may have a substantially same placement within the sheathing 303 at the first end 301 relative to the second end 304 (e.g., optical fiber 302a). In some embodiments, substantially all the optical fibers 302 may have substantially different placement within the sheathing 303 at the first end 301 relative to the second end 304.

In certain embodiments, the randomized arrangement found within the randomized fiber optic bundle 300 may allow for a more uniform distribution and/or mixing of light as it exits the second end 304. In certain embodiments, the first end 301 may be a fused end such that no adhesive is used to hold together each of the optical fibers 302. In some embodiments, the second end 304 may be an epoxied end such that an epoxy resin is used to hold together each of the optical fibers 302. In some embodiments, the first end 301 is a fused end or an epoxied end. In various embodiments, the second end 304 is a fused end or an epoxied end. In some embodiments, the randomized fiber optic bundle 300 is formed by twisting a fiber optic bundle.

In some embodiments, the optical fibers 302 may be randomized borosilicate fibers having a core diameter in a range of ˜15 to 100 μm (micrometers), ˜20 to 85 μm, and/or ˜30 to 70 μm. In certain embodiments, the core diameter may be ˜50 μm. In some embodiments, ˜1,000 to 2,000 optical fibers 302 may be packed to form an active area of ˜0.5 to 4 mm and are randomly arranged. In certain embodiments, ˜1,400 optical fibers 302 may be packed to form an active area of ˜2 mm. In certain embodiments, the sheathing 303 is a metal mono-coil and polyvinyl chloride sheathing. In various embodiments, the optical fibers 302 of the fiber optic bundle 300 may be borosilicate fibers with the numerical aperture in a range of ˜0.22 to 0.85 which are fused on the first end 301 and epoxied on the second end 304. In some embodiments, the numerical aperture may be ˜0.55. In some embodiments, the first end 301 may be fused and have an active area diameter in a range of ˜0.5 to 2.5 mm. In certain embodiments, the active area diameter may be ˜1.9 mm. In certain embodiments, the second end 304 may be epoxied and have an active diameter in a range of ˜1.0 to 3.0 mm. In some embodiments, the second end 304 may be epoxied and have a ˜2.05 mm active area diameter.

FIGS. 4A-4C illustrate example embodiments of fiber optic bundles that may serve, for example, as the first plurality of light carriers 107 of FIGS. 1-2. FIG. 4A shows a fiber optic bundle 401 having a split-rod 402. In some embodiments, the fiber optic bundle 401 may be a randomized fiber optic bundle (e.g., the fiber optic bundle 300). In some embodiments, the fiber optic bundle 401 may be an ordered fiber optic cable such that a one-to-one mapping exists between a light input end and a light output end. In some embodiments, the split-rod 402 is a split glass rod. In some embodiments, the split-rod 402 receives light at an input end 405 and outputs light via two output ends 406a and 406b. In certain embodiments, output ends 406a and 406b are each coupled to a homogenizer (e.g., as illustrated and described in FIG. 2).

FIG. 4B shows a bifurcated fiber optic bundle 400. In some embodiments, bifurcated fiber optic bundle 400 has an input end 410 where optical fibers are grouped together and subsequently branch out into a first fiber optic segment 412 and a second fiber optic segment 414. In certain embodiments, each of the first fiber optic segment 412 and the second fiber optic segment 414 may include a randomized fiber optic bundle (e.g., the fiber optic bundle 300). In certain embodiments, each of the first fiber optic segment 412 and the second fiber optic segment 414 may include an ordered fiber optic bundle. In certain embodiments, the first fiber optic segment 412 and the second fiber optic segment 414 are each coupled to a homogenizer.

FIG. 4C illustrates a fiber optic bundle 403 having a fiber optic splitter 404. In some embodiments, the fiber optic bundle 403 may be a randomized fiber optic bundle (e.g., the fiber optic bundle 300). In some embodiments, the fiber optic bundle 403 may be an ordered fiber optic cable such that a one-to-one mapping exists between a light input end and a light output end. In certain embodiments, the fiber optic bundle 403 is a fiber optic bundle with a single core. In embodiments illustrated in FIG. 4C, the fiber optic splitter 404 is a 1×2 fiber optic splitter. However, in other embodiments, the fiber optic splitter 404 may be a 1×n fiber optic splitter, where n is an integer greater than 1 (e.g., 1×2, 1×3, 1×4 . . . 1×n). In some embodiments, the fiber optic splitter 404 receives light at an input end 407 and outputs light from two output ends 408a and 408b. In certain embodiments, output ends 408a and 408b are each coupled to a homogenizer.

While FIG. 4C is described in relation to the fiber optic bundle 403 being split into two output ends, in some embodiments, multiple input ends may be combined to merge into one output end. In certain embodiments, an n×1 merging (e.g., 4×1) may be utilized to join optical fibers and/or fiber optic bundles into one fiber bundle. For example, a 3×1 merging may occur that merges fiber optics from a green, red, and blue light source into a single fiber optic bundle.

FIG. 5 illustrates an example embodiment of light carrier 500 that may serve, for example, as the second light carrier 201 of FIG. 2. Light carrier 500 includes a core 501 having a cladding 502 in contact with an outside surface of the core 501. Light 503 enters the light carrier 500 through input 504 and travels through the core 501 thus allowing propagation through the light carrier 500. Light 503 exits through the light carrier 500 via an output 505. In some embodiments, the light carrier 500 may have a cross-sectional (perpendicular to a main axis of the light carrier 500) shape that includes, without limitation, a hexagonal shape, a circular shape, a square/rectangular shape, or the like.

In some embodiments, the core 501 may include, for example, glass, air, silica, fused silica, or other material allowing the light 503 to propagate though the core 501. In certain embodiments, the cladding 502 may include, for example, a material with a surface in contact with the outside surface of the core 501. In certain embodiments, the surface may be mirrored or have a metallic coating thereon to provide mirror-like properties. In certain embodiments, the cladding 502 may include air. In various embodiments, the cladding 502 may include a material that has a lower refractive index than the core 501. In certain embodiments, the second light carrier may form a homogenizer to provide spatial homogenization and color mixing of the light 503. In certain embodiments, the light carrier 500 may be a clad-rod borosilicate homogenizer.

FIG. 6 illustrates an example embodiment of a light carrier 600 that may serve, for example, as the second light carrier 201 of FIG. 2. In the illustrated embodiment, the light carrier 600 has a hexagonal shape and includes a core 601 having cladding 602 in contact with an outer surface thereof. Light carrier 600 further includes an aperture 603 on a light output side thereof. In certain embodiments, the optical module 202 focuses light emitted from aperture 603 onto a target area, for example, the corneal plane. In some embodiments, the core 601 may be substantially similar to core 501 of FIG. 5. In certain embodiments, the cladding 602 may be substantially similar to cladding 502 of FIG. 5. While FIGS. 5-6 describe light carriers having a physical cladding layer (e.g., a clad rod light carrier), in some embodiments, the light carriers described herein may include light pipes and/or glass rods composed of fused silica, glass, silica, and the like. In such embodiments, the physical cladding layer is replaced by air (i.e., no physical cladding layer) but air effectively acts like a cladding layer in that it results in the confinement of the light within the core via total internal reflection.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those of ordinary skill in the art may better understand the aspects of the disclosure. Those of ordinary skill in the art should appreciate that they may readily use the disclosure as a basis for designing or modifying other processes and structures for carrying out the same purposes and/or achieving the same advantages of the embodiments introduced herein. Those of ordinary skill in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments.

While the above detailed description has shown, described, and pointed out novel features as applied to various embodiments, it will be understood that various omissions, substitutions, and changes in the form and details of the embodiments illustrated can be made without departing from the spirit of the disclosure. As will be recognized, the various embodiments described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. An optical system comprising:
a light source coupled to a plurality of first light carriers providing a plurality of light intensities;
a second light carrier coupled to the plurality of first light carriers, the second light carrier spatially homogenizing the plurality of light intensities to form a homogenized light intensity; and
an illumination module coupled to the second light carrier and operable to receive the homogenized light intensity.

2. The optical system of claim 1, wherein the plurality of first light carriers comprises a randomized fiber optic bundle.

3. The optical system of claim 2, wherein the randomized fiber optic bundle comprises a first end and a second end, the first end comprising fused optical fibers of the randomized fiber optic bundle and the second end comprising epoxied fibers of the randomized fiber optic bundle.

4. The optical system of claim 1, wherein the plurality of first light carriers comprises at least one of a fiber optic bundle comprising a split rod, a bifurcated fiber optic bundle, or a fiber optic bundle comprising one or more fiber splitters.

5. The optical system of claim 1, wherein the second light carrier comprises a cylindrical clad-rod borosilicate.

6. The optical system of claim 1, wherein the second light carrier comprises air.

7. The optical system of claim 1, wherein the second light carrier comprises a hexagonal rod comprising a round aperture.

8. The optical system of claim 1, wherein the second light carrier comprises at least one of a cylindrical shape, a hexagonal shape, a rectangular shape, or a square shape.

9. The optical system of claim 1, wherein the second light carrier comprises a cladding.

10. The optical system of claim 1, wherein the second light carrier comprises a mirrored surface.

11. The optical system of claim 1, wherein the illumination module comprises at least one of a lens or a beam splitter.

12. The optical system of claim 1, wherein the light source comprises at least one of a laser, an ultraviolet light source, an infrared (IR) light source, a near-IR light source, or one or more light-emitting diodes (LEDs).

13. The optical system of claim 12, wherein the second light carrier mixes light color from the plurality of light intensities.

14. An optical system comprising:
a light source coupled to a light carrier, the light carrier spatially homogenizing a plurality of light intensities to form a homogenized light intensity;
an optical head comprising an illumination module coupled to the light carrier and operable to receive the homogenized light intensity; and
wherein the optical head is spaced apart from the light source.

15. The optical system of claim 14, wherein:
the light source is coupled to a base of a microscope;
the optical head is coupled to a portion of an arm of the microscope; and
the portion of the arm is positioned over a corneal plane.
